(19) United States
(12) Patent Application Publication
(10) Pub. No.: US 2023/0015266 A1
(43) Pub. Date: Jan. 19, 2023

(54) NEONATAL FIBRIN SCAFFOLDS FOR PROMOTING WOUND HEALING

(71) Applicant: North Carolina State University, Raleigh, NC (US)

(72) Inventors: Ashley C. BROWN, Raleigh, NC (US); Kimberly A. NELLENBACH, Raleigh, NC (US)

(21) Appl. No.: 17/780,843

(22) PCT Filed: Nov. 30, 2020
(86) PCT No.: PCT/US2020/062580

§ 371 Date: May 27, 2022

Related U.S. Application Data
(60) Provisional application No. 62/942,001, filed on Nov. 29, 2019.

(51) Int. Cl. A61L 27/22; A61L 27/54

(52) U.S. Cl. CPC A61L 27/225; A61L 27/54; A61L 27/227; A61L 2400/12; A61L 2300/404; A61L 2300/402; A61L 2300/41

(57) ABSTRACT

Disclosed are methods of promoting wound healing in a patient in need thereof comprising administering to the patient a composition comprising a neonatal fibrin scaffold. Further disclosed are in vitro methods for evaluating a target composition on human wound healing comprising a neonatal porcine plasma scaffold with the target composition and evaluating scaffold properties of the plasma sample.

### CROSS-REFERENCE TO RELATED APPLICATION

This application claims the benefit of U.S. Provisional Application 62/942,001, filed Nov. 29, 2019, the contents of which are hereby incorporated in its entirety.

### ACKNOWLEDGEMENT OF GOVERNMENT SUPPORT

This invention was made with government support under grant number W81XWH-15-1-0485 awarded by the U.S. Army Medical Research and Development Command. The government has certain rights in the invention.

### FIELD OF THE INVENTION

The invention is directed to neonatal fibrin scaffolds which are useful for promoting wound healing. In an embodiment, the scaffolds are derived from neonatal porcine sources.

### BACKGROUND

Fibrin scaffolds are often utilized to treat chronic wounds. The monomer fibrinogen used to create such scaffolds is typically derived from adult human or porcine plasma. Wound healing outcomes have been linked to fibrin matrix structure, including fiber alignment, which can affect the binding and migration of cells.

However, there remains a need for improved methods of treating wounds, and there remains a need for improved fibrin scaffolds.

### SUMMARY

In accordance with the purposes of the disclosed materials and methods, as embodied and broadly described herein, the disclosed subject matter, in one aspect, relates to compounds, compositions and methods of making and using compounds and compositions. In specific examples, discloses are methods of promoting wound healing in a patient in need thereof comprising administering to the patient a composition comprising a neonatal fibrin scaffold.

The neonatal fibrin scaffolds may be used to treat wounds, by administering the scaffolds to the site of the wound. Exemplary wounds that may be treated with the neonatal fibrin scaffolds include a trauma wound, a surgical wound, a burn wound, or an ulcer wound. Further disclosed are in vitro and in vivo methods for evaluating a target composition on promoting wound healing. Compositions for use in the methods are also disclosed.

In an embodiment, the neonatal fibrin scaffold is a non-human derived fibrin scaffold. In some embodiments, the neonatal fibrin scaffold is a neonatal porcine fibrin scaffold. The neonatal fibrin scaffolds enhance wound healing outcomes compared to adult fibrin scaffolds.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts the quantification of fibrinogen levels in platelet poor plasma. Fibrinogen was quantified via ELISA across species and age ranges. N=3.

FIG. 2 depicts the quantification of isolated fibrinogen clottability across species. Clottability of purified fibrinogen was determined via NanoOrange Protein Quantification Kit. N=4 *p<0.05, **p<0.01

FIGS. 3A-3C depict confocal microscopy analysis of structure of adult and neonatal porcine scaffolds formed with increasing thrombin (FIG. 3A). Analysis of scaffold fiber alignment (FIG. 3B) and fiber density (FIG. 3C). 0.1 U/mL Thrombin: neonatal pigs N=4, adult pigs N=4, 0.25 U/mL Thrombin: neonatal pigs: N=8, adult pigs N=7, 1.0 U/mL Thrombin: neonatal pigs: N=8, adult pigs N=6. *p<0.05.

FIGS. 4A-4C depicts confocal microscopy analysis of structure of adult and neonatal human and porcine scaffolds formed with 0.5 U/mL Thrombin (FIG. 4A). Analysis of scaffold fiber alignment (FIG. 4B) and fiber density (FIG. 4C). Alignment: neonatal humans: N=4, adult humans: N=3, neonatal pigs: N=8, adult pigs: N=6. Fiber Density: neonatal humans: N=4, adult humans: N=3, neonatal pigs: N=5, adult pigs: N=3. *p<0.05, **p<0.01.

FIG. 5 depicts atomic force microscopy analysis of plasma scaffold stiffness across species. N=3; *p<0.05.

FIGS. 6A-C depict the evaluation of plasma scaffold degradation with a custom microfluidic device. Top view of the device (FIG. 6A) Scale=10 μm. FIG. 6B depicts an enlarged view of the clot juncture, showing the migration of the clot boundary. Initial (green) and final (red) frames of clot boundary overlaid with false coloring (B). In black and white rendering, the green frame is presented as a darker grey than the red frame. FIG. 6C depicts the average Degradation rates+/−standard deviation. neonatal humans: N=3, adult humans: N=3, neonatal pigs: N=5, adult pigs: N=5, Scale=10 μm, *p<0.05, **p<0.01, ***p<0.001, ****p<0.0001.

FIG. 7 depicts a CryoSEM analysis of adult and neonatal porcine plasma scaffolds at 2500×. 50 μL scaffolds were firmed with 0.5 U/mL thrombin and polymerized for two hours prior to imaging. Representative images are shown. Scale=10 μm, N=3.

FIG. 8 depicts representative atomic force microscopy forcemaps of adult and neonatal porcine and human scaffolds. N=3.

FIG. 9 depicts that neonatal and adult fibrin scaffolds are structurally distinct. (A) Representative confocal images taken at 63× of adult or neonatal fibrin scaffolds polymerized with 2.5 mg/mL fibrinogen and 0.5 U/mL thrombin. (B) Fiber density was calculated as the ratio of black (fibers) over white (space) fibers. (C) Alignment index and (D) quantification of branch points was conducted with custom MATLAB codes. N=3 scaffolds per group with 3 random images each. Mean+/−standard deviation is shown. Scale=10 μm. p**<0.01, p***<0.001.

FIG. 10 depicts that fibroblast attachment is greater on neonatal fibrin scaffolds. Fluorescently labeled HDFns were seeded on adult or neonatal derived fibrin scaffolds and attachment was investigated after 1 hour via fluorescent plate reader. Mean attachment+/−standard deviation is shown. N=6 wells per group. p**<0.01.

FIG. 11 depicts that fibroblast attachment is similar on desalinated adult and neonatal fibrin scaffolds. Sialic acid was cleaved via neuraminidase digestion and then fibroblast attachment on desalinated fibrin scaffolds was investigated. Fluorescently labeled HDFns were seeded on adult or neonatal derived desalinated fibrin scaffolds and attachment was investigated after 1 hour via fluorescent plate reader. Mean attachment+/−standard deviation is shown. N=6 wells per group.

FIG. 12 depicts that fibroblast spreading is greater on neonatal fibrin films. (A) Representative confocal microscopy images of fibroblast morphology on neonatal or adult fibrin films at 40×. Cells were seeded on fluorescently labeled fibrin networks (purple) at a density of 6,000 cells per well for 16 hours prior to fixation. Fluorescent phalloidin (green) was used for membrane visualization. (B) Cell area, (C) perimeter, and (D) circularity was quantified with ImageJ. Mean+/−standard deviation is shown. Scale=10 μm. p****<0.0001. N=15 cells per group.

FIG. 13 depicts that fibroblast migration is accelerated through neonatal fibrin scaffolds compared to adult fibrin scaffolds. (A) Schematic of spheroid migration assay utilized to characterize fibroblast migration through 3D fibrin scaffolds. HDFns are cultured into spheroids and subsequently embedded in a 3D fibrin scaffold. Migration away from the spheroid body is measured daily for 72 hours and is quantified by measuring the spheroid boundary using Image J. (B) Representative images of cell migration outward from the spheroid after 72 hours imaged at 10×. Fibroblast outgrowth is outlined in white. (C) Migration results over 72 hours. Mean areas+/−standard deviation is shown. Scale=200 μm. N=3-5/group. p***<0.001.

FIG. 14 depicts that wound closure is accelerated in the presence of neonatal fibrin scaffolds compared to adult fibrin scaffolds. (A) Neonatal or adult derived fibrin scaffolds were applied to a rodent full thickness dermal injury and wound healing was monitored over 9 days. Representative images of wounds treated with neonatal fibrin, adult fibrin, or saline on day 0, 4, and 9, (B) % wound closure over 9 days and (C) wound healing rate are shown. Means+/−standard deviation are shown. N=6 wounds per group. p*<0.05.

FIG. 15 depicts that neonatal fibrin scaffolds enhance epidermal thickness and angiogenesis compared to adult fibrin scaffolds. (A) Representative images of wounds stained with MSB and CD31 at 10×. FIGS. 15B and 15C depict quantification of images was performed by measuring thickness of epidermal layer in ImageJ and using ImageJ particle analysis to measure CD31 (red) area. MSB staining revealed significantly greater epidermal thickness in wounds treated with neonatal fibrin scaffolds. N=5-6. Immunolabeling for CD31+ tissue suggests enhanced, though not statistically significant, angiogenesis in wounds treated with neonatal fibrin. N=4-6. Means+/−standard deviation is shown. p*<0.05.

FIG. 16 depicts that fibroblast attachment is greater on neonatal fibrin scaffolds. HDFns were seeded on neonatal or adult derived fibrin scaffolds at a density of 12,000 cells per well and incubated for 16 hours prior to fixation. Cells were labeled fluorescently labeled for visualization on confocal microscopy. Cell count was quantified as number of cells per viewing area. Mean cell count+/−standard deviation is shown. N=12 images per group. P*<0.05.

FIG. 17 depicts that fibroblast spreading is greater on neonatal fibrin films. (A) Representative confocal microscopy images of fibroblast morphology on neonatal or adult fibrin films at 40×. Cells were seeded on fibrin networks at a density of 12,000 cells per well and incubated for 16 hours prior to fixation. Fluorescent phalloidin (green) was used for membrane visualization. (B) Cell area, (C) perimeter, and (D) circularity was quantified with ImageJ. Mean values+/−standard deviation are shown. N=20 cells per group. Scale=10 μm. p*<0.05, p****<0.0001.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred. or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein, the term fibrin scaffold refers to a polymerized fibrinogen, which can in some instances be referred to as a clot. The scaffold may be in bulk form, or may be in the form of particles.

As used herein, the term “platelet poor” refers to a composition that is essentially free of platelets.

As used herein, the phrase “therapeutic agent” refers to any agent that, when administered to a subject, has a therapeutic and/or diagnostic effect and/or elicits a desired biological and/or pharmacological effect.

As used herein, the term “wound” refers to physical disruption of the continuity or integrity of tissue structure. Wounds may be acute or chronic and include cuts and lacerations, surgical incisions or wounds, punctures, grazes, scratches, compression wounds, abrasions, friction wounds, decubitus ulcers (e.g. pressure or bed sores); thermal effect wounds (burns from cold and heat sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g., psoriasis and eczema, stomach or intestinal ulcers, oral wounds, including a ulcers of the mouth, damaged cartilage or bone, amputation wounds and corneal lesions.

As used herein, the term “chronic wound” refers to a wound that has not healed within a normal time period for healing in an otherwise healthy subject. Chronic wounds may be those that do not heal because of the health of the subject, for example, where the subject has poor circulation or a disease such as diabetes, or where the subject is on a medication that inhibits the normal healing process. Healing may also be impaired by the presence of infection, such as a bacterial, fungal or parasitic infection. In some instances, a chronic wound may remain unhealed for weeks, months or even years. Examples of chronic wounds include but are not limited to, diabetic ulcers, pressure sores and tropical ulcers (i.e., jungle rot).

Extensive differences have been observed in fibrin network properties between adults and neonates, including higher fiber alignment in neonatal networks. Wound healing outcomes have been linked to fibrin matrix structure, including fiber alignment, which can affect the binding and migration of cells. It is hypothesized that fibrin scaffolds derived from neonatal fibrin would enhance wound healing outcomes compared to adult fibrin scaffolds. Fibrin scaffolds can be formed from purified adult or neonatal fibrinogen and thrombin. Significantly higher fibroblast attachment and migration was observed on neonatal scaffolds compared to adults. Cell morphology on neonatal scaffolds exhibited higher spreading compared to adult scaffolds. In vivo significantly smaller wound areas and greater epidermal thickness were observed when wounds were treated with neonatal fibrin compared to adult fibrin or a saline control. Fibrin scaffolds sourced from neonatal plasma improve healing outcomes compared to scaffolds sourced from adult plasma. Additionally, we demonstrate that neonatal fibrinogen sourced from porcine plasma is compatible with human tissue, therefore, neonatal fibrin scaffolds sourced from non-human neonatal fibrinogen can be useful for creating pro-healing scaffolds.

Disclosed herein are fibrin scaffolds comprising crosslinked neonatal fibrin. The fibrin scaffolds can be prepared from polymerizing neonatal fibrinogen with thrombin or other appropriate crosslinking agent. In some embodiments, the fibrin scaffolds are prepared by polymerizing a neonatal fibrinogen, e.g., neonatal porcine fibrinogen, composition having a concentration no greater than 100 mg/ml, no greater than 50 mg/mL, no greater than about 25 mg/ml, no greater than about 10 mg/ml, no greater than about 5 mg/ml, no greater than about 2.5 mg/ml, no greater than about 1.0 mg/ml, no greater than about 0.5 mg/ml, or no greater than about 0.1 mg/ml. In some instances, the neonatal fibrinogen, e.g., neonatal porcine fibrinogen composition can have a density between about 0.1-100 mg/ml, between about 0.1-10 mg/ml, between about 0.1-5 mg/ml, between about 0.1-1.0 mg/ml, between 1-10 mg/ml, or between about 1-5 mg/ml. The composition may be polymerized by mixing with thrombin in a concentration of at least 0.05 U/ml, at least 0.1 U/ml, at least 0.25 U/ml, at least 0.5 U/ml, at least 0.75 U/ml, or at least 1.0 U/ml, or at least 10 U/ml. In some embodiments, the composition may be polymerized by mixing with thrombin in a concentration between 0.05-10 U/ml, between 0.1-1 U/ml, between 0.25-1 U/ml, or between 0.25-0.75 U/ml, between 1-10 U/ml, between 1-5 U/ml, between 5-10 U/ml, between 0.25-5 U/ml, or between 0.25-2.5 U/ml.

In certain embodiments, the neonatal fibrinogen is obtained from an animal no more than 18 months of age, no more than 12 months of age, no more than 10 months of age, no more than 8 months of age, no more than 6 months of age, no more than 4 months of age, no more than 2 months of age, no more than 1 month of age, or no more than two weeks of age. In some embodiments, the neonatal fibrinogen is obtained from an animal between 1-12 months of age, between 1-10 months of age, between 1-8 months of age, between 1-6 months of age, between 1-4 months of age, or between 1-3 months of age.

In some embodiments, the neonatal fibrinogen is obtained by centrifuging blood samples to obtain platelet poor plasma that is essentially free of cellular components. In some embodiments, the platelet poor plasma can be used to generate purified fibrinogen via a precipitation procedure. In certain embodiments, the animal is a pig, for instance a Duroc pig, a Berkshire pig, a Yorkshire pig, a Spotted pig, a Landrace pig, a Poland China pig, a Hampshire pig, or a Chester White pig.

In certain embodiments, the fibrinogen used to prepare the fibrin scaffolds can have a clottability that is less than 99, less than 90, less than 50, less than 45, less than 40, less than 35, less than 30, less than 25, less than 20, less than 15, less than 10, or less than 5. In some instances, the fibrin scaffolds can have a clottability that is between 5-99, 5-50, between 5-25, between 5-10, between 10-25, between 20-40, between 25-50, between 25-75, between 75-99, between 25-99, or between 50-99. Clottability may be determined using ELISA, for instance NanoOrange Protein Quantification Kit (Invitrogen, USA).

In some instances the fibrin scaffolds can be characterized by a stiffness that is less that 5 k*pa, less than 4 k*Pa, less than 3 k*Pa, less than 2.5 k*Pa, less than 2 k*Pa, less than 1.5 k*Pa, less than 1 k*Pa, less than 0.5 k* Pa, less than 0.25 k*Pa, less than 0.1 k*Pa, or less than 0.05 k*Pa.

In some embodiments the fibrin scaffolds can be characterized by a fiber density of less than 2, less than 1.75, less than 1.5, less than 1.25, less than 1, less than 0.9, less than 0.8, less than 0.7, less than 0.6, less than 0.5, less than 0.4, less than 0.3, less than 0.2, or less than 0.1. In some embodiments, the fibrin scaffolds have a fiber density between 0.1-2, between 0.1-1.5, between 0.1-1, between 0.1-0.8, between 0.1-0.6, between 0.1-0,4, between 0.2-1, between 0.2-0.8, between 0.4-1, between 0.5-2, between 0.5-1.5, or between 1-2. Fiber density is the ratio of black pixels (fibers) over white pixels (blank space) in confocal microscopy images.

In certain embodiments the fibrin scaffolds can be characterized by an alignment index of at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, or at least 1.9. Alignment index (AI) is determined from the fraction of fibers aligned within +/−20 degrees of a preferred fiber alignment normalized to random distribution of oriented fibers.

In some instances, the fibrin scaffolds have a density no greater than 50 mg/ml, no greater than about 25 mg/ml, no greater than about 10 mg/ml, no greater than about 5 mg/ml, no greater than about 2.5 mg/ml, no greater than about 1.0 mg/ml, no greater than about 0.5 mg/ml, or no greater than about 0.1 mg/ml. In some instances, the fibrin scaffolds can have a density between about 0.1-25 mg/ml, between about 0.1-10 mg/ml, between about 0.1-5 mg/ml, between about 0.1-1.0 mg/ml, between 1-10 mg/ml, or between about 1-5 mg/ml.

In some embodiments, the fibrin scaffolds can be formulated into fibrin based particles that have an average particle size no greater than about 10,000 nm, no greater than about 5,000 nm, no greater than about 2,500 nm, no greater than about 1,000 nm, no greater than about 750 nm, no greater than about 500 nm, or no greater than about 250 nm. In some embodiments, the particles can have an average particle size between about 100-10,000 nm, between about 100-5,000 nm, between about 100-2,500 nm, between about 250-2,500 nm, between about 500-2,500 nm, between about 1,000-2,500 nm, between about 100-1,500 nm, between about 100-1,000 nm, between about 100-750 nm, between about 100-500 nm, or between 100-250 nm.

The fibrin particles disclosed herein are porous and have hydrogel properties. For instance, when dehydrated the particles have a substantially flat shape, but when suspended in water attain a bulbous, more spherical shape. For instance, when dehydrated, the particles may have a height that is no more than 20%, no more than 15%, no more than 10%, no more than 8%, no more than 6%, no more than 4%, or no more than 2% of the width.

In some embodiments, the fibrin particles do not exhibit a substantial degree of covalent crosslinking. For instance, the particles may have a degree of covalent crosslinking no greater than 25%, no greater than 20%, no greater than 15%, no greater than 10%, no greater than 5%, no greater than 2.5% or no greater than 1%. In some embodiments, the particles have a degree of crosslinking between 1-25%, between 1-20%, between 1-15%, between 1-10%, between 1-5%, between 1-2.5%, between 2.5-10%, between 5-15%, or between 10-25%. Crosslinking may be evaluated using the technique disclosed in U.S. Pat. No. 6,150,505, e.g., col. 3, line 41-55, and col. 9, line 52-col. 10, line 11, incorporated herein by reference.

The neonatal fibrin scaffolds described in each of the aforementioned paragraphs can further include at least one therapeutic agent. Exemplary therapeutic compounds include antimicrobials, analgesics and anti-inflammatories. In some cases, the therapeutic compound is an antimicrobial agent, e.g., an agent that inhibits the growth of or kill microbes such as bacteria, mycobacteria, viruses, fungi, and parasites. Anti-microbial agents therefore include anti-bacterial agents, anti-mycobacterial agents, anti-viral agents, anti-fungal agents, and anti-parasite agents. Suitable antimicrobials include antibiotics, analgesics, antimicrobial peptides and metallic compounds. Suitable analgesics include opioids, capsaicin, diclofenac, lidocaine, benzocaine, methyl salicylate, trolamine, prilocaine, prarnoxine, dibucaine, phenol, tetracaine, camphor, dyclonine, and menthol. Suitable anti-inflammatories include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, chprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, dillumidone sodium, diflunisal, difluprednate, diftalone, dimethyl sulfoxide, drocinonide, endrysone, enlimomab, enolicam, sodium, epirizole, etodolac, etofenamate, felbinac, fenamole, fenbufen, fenclofenac, fenclorac, fendosal, fenpipalone, fentiazac, flazalone, fluazacort, flufenamic acid, flurnizole, flunisolide acetate, flunixin, flunixin meglumine, fluocortin butyl, fluorometholone acetate, fluquazone, flurbiprofen, fluretafen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, semietacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, tricionide, triflumidate, zidometacin, and zomepirac sodium.

In some cases, the therapeutic agent can include at least one growth factor, cytokine, chemokine, CD antigen, neutrophin, hormone, enzyme, viral antigen, bacterial antigen, recombinant protein, natural protein, monoclonal antibody, polyclonal antibody, donor blood serum protein, donor blood plasma protein, or small molecule drug. Exemplary growth factors include KGF, PDGF, TGF_{β}, interleukin, activin, colony stimulating factor, CTGF, EGF, Epigen, erythropoietin, FGF, galectin, HDGF, hepatocyte growth factor, IGFBP, insulin like growth factor, insulin, leptin, macrophage migration inhibitory factor, melanoma inhibitory factor, myostatin, noglzin, NOV, omentin, oncostatinM, osteopontin, OPG, periostin, placenta growth factor, placental lactogen, prolactin, RANK ligand, retinol binding protein, stem cell factor, transforming growth factor, and VEGF. In certain preferred embodiments, the scaffolds described in the above paragraphs can include KGF, IL-2, and/or IL-6.

Therapeutic agents, such as described above, can be covalently conjugated to the fibrin molecules in the nanoparticle. For instance, the therapeutic agent can be conjugated to the surface of the particle, or can be within the volume of the nanoparticle. In further embodiments, a therapeutic agent can be entrapped or encapsulated within the nanoparticle, for instance, not covalently bonded to the fibrin molecules.

Therapeutic agents may be conjugated to the fibrin molecules through a linker. Any suitable linker can be used in accordance with the present invention. Linkers may be used to form amide linkages, ester linkages, disulfide linkages, etc. Linkers may contain carbon atoms or heteroatoms (e.g., nitrogen, oxygen, sulfur, etc.). Typically, linkers are 1 to 50 atoms long, 1 to 40 atoms long, 1 to 25 atoms long, 1 to 20 atoms long, 1 to 15 atoms long, 1 to 10 atoms long, or 1 to 10 atoms long. Linkers may be substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

A linker can an aliphatic or heteroaliphatic linker. For example, the linker can a polyalkyl linker. The linker can be a polyether linker. The linker can be a polyethylene linker, such as PEG. The linker can be a short peptide chain, e.g., between 1 and 10 amino acids in length, e.g., 1, 2, 3, 4, or 5 amino acids in length, a nucleic acid, an alkyl chain, etc.

The linker can be a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g. esterase cleavable linker), ultrasound-sensitive linkers, x-ray cleavable linkers, etc. In some embodiments, the linker is not a cleavable linker.

Any of a variety of methods can be used to associate a linker with a particle and agent. General strategies include passive adsorption (e.g., via electrostatic interactions), multivalent chelation, covalent bond formation, etc. (Gao et al, 2005, Curr. Op. Biotechnol., 16:63). Click chemistry can be used to associate a linker with an agent (e.g. Diels-Alder reaction, Huigsen 1,3-dipolar cycloaddition, nucleophilic substitution, carbonyl chemistry, epoxidation, dihydroxyrlation, etc.).

A bifunctional cross-linking reagent can be employed. Such reagents contain two reactive groups, thereby providing a means of covalently associating two target groups. The reactive groups in a chemical cross-linking reagent typically belong to various classes of functional groups such as succinimidyl esters, maleimides, and pyridyldisulfides. Exemplary cross-linking agents include, e.g., carbodiimides, N-hydroxysuccinimidyl-4-azidosalicylic acid (NHS-ASA), ditnethyl pimelimidate dihydrochloride (DMP), dimethylsuberimidate (DMS), 3,3′-dithiobispropionimidate (DTBP), N-Succinimidyl 3-[2-pyridyldithio]-propionamido (SPDP), succimidyl α-methylbutanoate biotinamidohexanoyl-6-amino-hexanoic acid N-hydroxy-succinimide ester (SMCC), succinimidyl-[(N-maleimidopropionamido)-dodecaethyleneglycol] ester (NHS-PEO 12), etc. For example, carbodiimide-mediated amide formation and active ester maleimide-mediated amine and sulfhydryl coupling are widely used approaches.

Common schemes for forming a conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g., a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to particles via amine or thiol groups in lysine or cysteine side chains respectively, or by an N-terminal amino group. Nucleic acids such as RNAs can be synthesized with a terminal amino group. A variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) may be used to associate the various components of conjugates. Agents can be prepared with functional groups, e.g., amine or carboxyl groups, available at the surface to facilitate association with a biomolecule. Any biomolecule can be attached to another molecule described herein using any of the methods described herein.

The scaffolds described above can be included in a wide variety of pharmaceutical compositions, for instance those including pharmaceutically acceptable carriers. Suitable carriers include water, saline and other liquid formulations, which can be directly administered to a wound site or injected into a patient. In other cases, the scaffolds can be included in a formulation for topical administration, for instance, lotions, sprays, creams, ointments and the like. The compositions can also include a backing layer to secure the composition at a wound site. In other embodiments, a first composition containing platelet poor fibrinogen may be contacted with a second composition containing thrombin at the site of a wound, in order to directly prepare the fibrin scaffold on the wound itself.

The pharmaceutical compositions described herein may be prepared by any method known or hereafter developed in the art of pharmaceutics. In general, such preparatory methods include the step of bringing the active ingredient into association with one or more excipients and/or one or more other accessory ingredients, and then, if necessary and/or desirable, shaping and/or packaging the product into a desired single- or multi-dose unit.

Dosage forms for topical and/or transdermal administration of the scaffolds may include ointments, pastes, creams, lotions, gels, powders, solutions, sprays, inhalants and/or patches. Generally, the active component is admixed under sterile conditions with a pharmaceutically acceptable excipient and/or any needed preservatives and/or buffers as may be required. Additionally, the present invention contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the excipients and/or additional ingredients described herein.

Pharmaceutically acceptable excipients used in the manufacture of pharmaceutical compositions include, but are not limited to, inert diluents, dispersing and; or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxy ethylene sorbitan [Tween 60], polyoxy ethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Plutonic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hvdroxytoluene, monothioglycerol, potassium metabisulfite, propionic acid, propyl gallate, sodium ascorbate, sodium bisulfate, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulftte, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate butler solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium laurel sulfate, sodium lauryl sulfate, etc., and combinations thereof.

Exemplary oils include, but are not limited to, almond, apricot kernel, avocado, babassu, bergamot, black current seed, borage, cage, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl ntyristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and perfuming agents. In certain embodiments for parenteral administration, conjugates can be mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U. S. P. and isotonic sodium chloride solution, etc. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the active ingredient then depends upon its rate of dissolution which, in turn, may depend upon crystal size and crystalline form. In some embodiments, delayed absorption of a parenterally administered active ingredient is accomplished by dissolving or suspending the drug in an oil vehicle.

Compositions for rectal or vaginal administration are typically suppositories which can be prepared by mixing the conjugates with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such sold dosage forms, the active ingredient is mixed with at least one inert, pharmaceutically acceptable excipient such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Spray or aerosol formulations may include one or more propellants. Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 μm to about 200 μm.

Also disclosed are methods of treating a wound by administering the scaffolds described above to the site the wound. Exemplary wounds that may be treated with the scaffolds include trauma wound, a surgical wound, a burn wound, or an ulcer wound. Wound patients with coagulation disorders may be advantageously treated with the scaffolds, for instance, diabetics, hemophiliacs, patients with vitamin K deficiency, Von Willebrand disease or other clotting factor deficiencies. The scaffolds can be used to treat wounds in patients undergoing anti-coagulation therapy, for instance patients receiving heparin, fandaparinux, idraparinux, vitamin K, coumadin, direct thrombin inhibitors like argatroban, dabigatran, factor Xa inhibitors like rivaroxaban, apixaban and edoxaban, anti-platelet agents such as clopidogrel and prasugrel. The scaffolds can be used to treat wounds arising from medical procedures such as stent placement, transfusion, and dialysis.

Disclosed is a comparative study demonstrating that neonatal fibrin scaffolds promote enhanced cell adhesion, migration, and wound healing in vivo compared to adult fibrin scaffolds. Fibrin scaffolds are often utilized to treat chronic wounds. The monomer fibrinogen used to create such scaffolds is typically derived from adult human or porcine plasma. However, disclosed here are studies that have identified extensive differences in fibrin network properties between adults and neonates, including higher fiber alignment in neonatal networks. Wound healing outcomes have been linked to fibrin matrix structure, including fiber alignment, which can affect the binding and migration of cells. It is hypothesized that fibrin scaffolds derived from neonatal fibrin would enhance wound healing outcomes compared to adult fibrin scaffolds. Disclosed herein are studies demonstrating that distinctions in neonatal and adult fibrin scaffold properties influence cellular behavior and wound healing. These studies indicate that fibrin scaffolds sourced from neonatal plasma could improve healing outcomes compared to scaffolds sourced from adult plasma.

Also, disclosed is a comparative study between neonatal and adult human and porcine plasma samples to determine if piglets accurately reflect the maturation of human fibrinogen to demonstrate that neonatal fibrinogen could be sourced from porcine plasma sources in addition to human plasma sources. Pigs represent an appealing model to use in translational medicine due to their anatomical and physiological similarities to humans particularly in regard to the hemostatic system (Münster A-M B, et al., “Usefulness of human coagulation and fibrinolysis assays in domestic pigs,” Comp Med 2002; 52:39-43; Zentai C, et al., “Fibrin patch in a pig model with blunt liver injury under severe hypothermia,” J Surg Res 2014, 187:616-24; Inaba K, et al., “Dried Platelets in a Swine Model of Liver Injury,” Shock 2014; 41:429-34). Thus, it is hypothesized that pigs possess age-related differences in fibrinogen that parallel those identified in humans (Greek R, et al., “Animal models and conserved processes,” Theor Biol Med Model 2012; 9:40). The primary objective of this study was to validate the fibrin network in piglets as an appropriate in vitro animal model for that of human neonates. This was accomplished by characterizing fibrin network parameters formed from plasma collected from neonatal and adult humans and pigs, If confirmed, piglets could serve as a suitable and much needed source for neonatal plasma.

### EXAMPLES

The following examples are for the purpose of illustration of the invention only and are not intended to limit the scope of the present invention in any manner whatsoever.

### Example 1: Comparison of Fibrin Scaffolds Derived from Human Blood with Fibrin Scaffolds Derived from Porcine Blood

After IRB approval and informed written consent, blood samples were collected from 10 human neonates (less than 30 days of age) undergoing corrective cardiac surgery with CPB at the Children's Hospital of Atlanta. All samples were collected from an arterial line placed after the induction of anesthesia and prior to surgical incision and CPB. Samples were centrifuged immediately to yield platelet poor plasma (PPP) and stored at −80° C. until use. All patient samples were deidentified prior to sample transfer to NCSU. Pooled adult human PPP was obtained from the New York Blood Center.

Blood was collected from 8 1-year old female Yorkshire pigs and 8-week-old Yorkshire piglets prior to planned surgical procedures at North Carolina State University's (NCSU) School of Veterinary Medicine, (Raleigh, N.C., USA) through the tissue sharing program. Porcine ages and sample size were selected in order to minimize animal use. For this study, samples from pigs that were readily available in sufficient numbers were utilized. All samples were collected via jugular venous puncture after the induction of anesthesia and prior to surgical incision. Samples were centrifuged immediately to obtain platelet poor plasma and stored at −80° C. until use.

Quantification of fibrinogen concentration was achieved via enzyme linked immunosorbent assays (ELISA; Abcam, USA) with human and porcine protein quantification kits. Isolated fibrinogen was utilized for clottability assays. Fibrinogen was purified from plasma via an ethanol precipitation reaction. Briefly, ethanol (70% volume) was added to 4° C. plasma in a 4:1 ratio (plasma:ethanol) and cooled on ice for 20 min. The solution was then centrifuged for 15 min at 4° C. The supernatant plasma was removed and the resulting pellet was heated in a 37° C. water bath. A buffer consisting of 20 mM sodium citrate was added until the pellet was fully dissolved. Protein concentration was determined via absorbance readings at 280 nm using a Nanodrop.

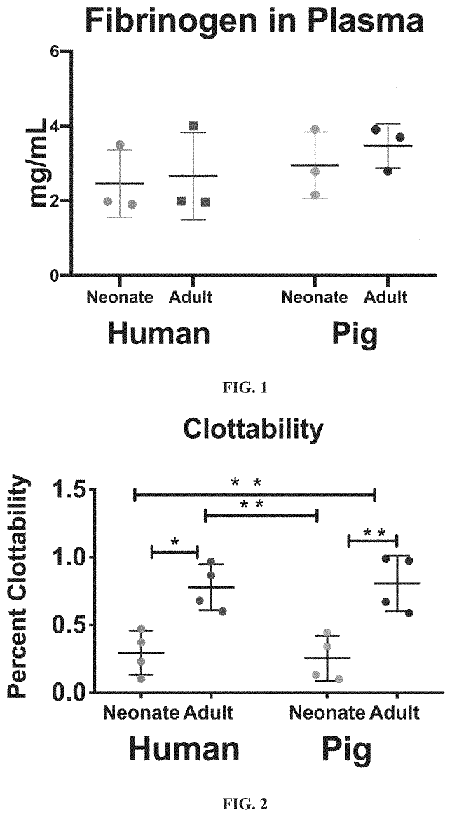

Fibrinogen concentrations were quantified across age groups and species (FIG. 1). Similar values of fibrinogen were observed across species and age groups that is consistent with previous studies (Neonatal humans: 246+/−90 mg/dL, neonatal pigs: 295+/−89 mg/dL, adult humans: 265+/−116 mg/dL, adult pigs: 346+/−0.59 mg/dL; neonatal humans vs. adult humans: P=0.993, neonatal pigs vs. adult pigs: P=0.898, neonatal human vs. neonatal pigs: P=0.909, adult human vs. adult pigs: P=0.705). Additionally, porcine fibrinogen concentrations were within normal human ranges of 200-450 mg/dL.

Percentage of total fibrinogen clottability was determined by a protein quantification-based assay which measures the protein content in the clot liquor (soluble portion of clot sample) remaining after polymerization. 50 μL scaffolds with purified fibrinogen at a concentration of 2.5 mg/mL, HEPES buffer (5 mM calcium, 7.4 pH) and were formed with the initiation of 0.5 U/mL thrombin. Five μL aliquots were taken before and after a one-hour polymerization period and quantified via NanoOrange Protein Quantification Kit (Invitrogen, USA). Alternately, 50 μL plasma scaffolds were formed with 0.5 U/mL thrombin and quantification was conducted via ELISA for pig or human fibrinogen (Abcam, USA). Percent of clottable fibrinogen was determined as: [(initial soluble protein)−(soluble protein in clot liquor)]/(initial soluble protein)×100.

To compare functional ability of fibrinogen across species and age groups, clottability of the purified protein was quantified (FIG. 2). It was demonstrated that neonatal samples had statistically significant lower clottability across species compared to their adult counterparts (Neonatal humans: 29+/−16%, neonatal pigs: 25+/−17%, adult humans: 78+/−17%, adult pigs: 81+/−21%; neonatal humans vs. adult humans: P=0.01, neonatal pigs vs. adult pigs: P=0.004). Neonatal humans and pigs had similar clottability (P=0.989) values as did adult samples (P=0.995).

Confocal microscopy was utilized to examine scaffold structure from neonatal and adult human and porcine plasma samples. 50 μL scaffolds consisting of 90% plasma by volume were polymerized with 0.1, 0.25, or 0.5 U/ml of human thrombin (Enzyme Research Labs, USA), and 10 μg/mL of Alexa-Flour 488 labeled fibrinogen for visualization. Scaffolds were formed between a glass slide and coverslip and allowed to polymerize for two hours prior to imaging. A Zeiss Laser Scanning Microscope (LSM 710, Zeiss Inc., USA) at a magnification of 63× was utilized for imaging and a minimum of three random 5.06 μm z-stacks were acquired per scaffold. ImageJ software was used to create 3D projections from z-stacks. Scaffold fiber density was determined from the ratio of black (fiber) over white (background) pixels in each image. Fibrin scaffold alignment was quantified with a custom matlab algorithm disclosed in Am J Physiol Heart Circ Physiol 2010; 298:NaN—NaN, the contents of which are hereby incorporated by reference. An alignment index (AI) was determined from the fraction of fibers aligned within +/−20 degrees of a preferred fiber alignment normalized to random distribution of oriented fibers. A greater AI corresponds to a higher percentage of fibers aligned near the preferred fiber alignment. AI values range from 1.0 to 4.55. Alignment analysis was conducted for each image in the z stack and averaged together. Scaffold structure in porcine samples was additionally assessed with cryogenic scanning electron microscopy (cryoSEM) to examine three-dimensional scaffold architecture. Again, 50 μL plasma scaffolds were formed with 0.5 U/mL thrombin and allowed to polymerize for two hours prior to imaging. Scaffolds were rapidly frozen in sub-cooled liquid nitrogen and imaged at 2,500×. Three scaffolds were imaged per group and three random images were taken per scaffold.

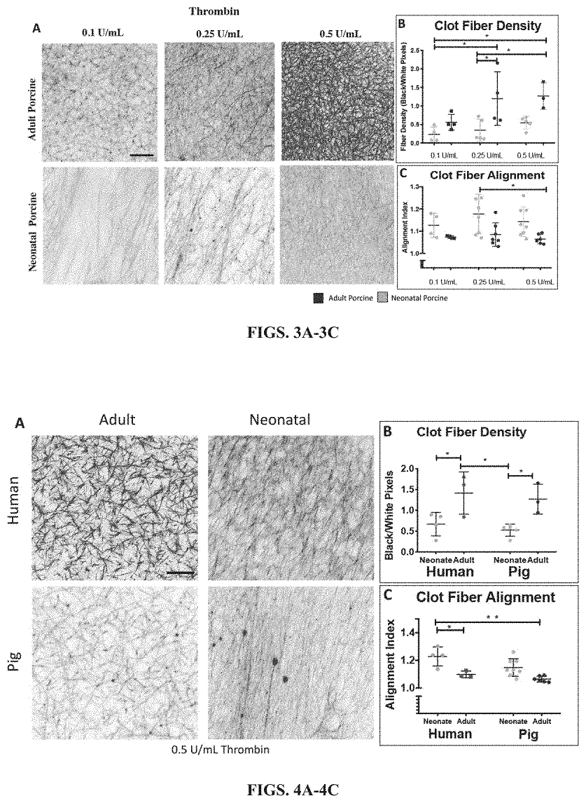

The fibrin scaffold structure was first examined and contrasted with confocal microscopy between adult and neonatal porcine samples as a function of thrombin concentration (FIGS. 3A-3C). In general, adult porcine scaffolds were denser and heavily branched with of increasing fiber density with increasing thrombin concentration. Fiber density is calculated as the ratio of black pixels (fibers) over white pixels (blank space), therefore this measurement is unitless. Conversely, neonatal porcine fibrin scaffolds were more aligned with a lower fiber density compared to adults (AI values: neonatal pigs 0.1, 0.25, and 0.5 U/mL thrombin: 1.12+/−0.05, 1.18+/−0.08, 1.15+/−0.09; adult pigs 0.1, 0.25, 0.5 U/mL thrombin: 1.07+/−0.001, 1.08+/−0,05, 1.07+/−0.07, 0.1 thrombin: neonatal pigs vs. adult pigs P=0.814, 0.25 thrombin: neonatal pigs vs. adult pigs P=0.063, 0.5 thrombin: neonatal pigs vs. adult pigs P=0.196; Fiber density: neonatal pigs 0.1, 0.25, 0.5 U/mL thrombin: 0.23+/−0.19, 0.25+/−0.29, 0.54+/−0.16; adult pigs 0.1-0.5 U/mL thrombin: 0.56+/−0.21, 1.19+/−0.72, 1.27+/−0.36, 0.1 thrombin: neonatal pits vs. adult pigs P=0.801, 0.25 thrombin: neonatal pigs vs. adult pigs P=0.026, 0.5 thrombin: neonatal pigs vs. adult pigs P=0.118). For all subsequent structural analyses, a thrombin concentration of 0.5 U/mL was utilized. Fibrin scaffold structure was also examined and contrasted with cryoSEM between neonatal and adult porcine samples (FIG. 7). Three-dimensional structure reflected similar structural patterns identified via confocal microscopy with neonatal porcine samples more highly aligned compared to the densely-branched network in adult porcine scaffolds. However, because the freezing technique required for cryoSEM may impact scaffold porosity, it was utilized as a secondary method of image analysis and did not include quantitative analysis. Next, confocal microscopy was utilized to examine fibrin scaffold architecture across species (FIGS. 4A-4C). In general, age-related structural relationships were consistent across species. Neonatal porcine and human scaffolds exhibited minimally branched, aligned, sheet-like fibrin matrices with significantly lower fiber densities than the adult groups (neonatal humans: 0.61+/−0.28, neonatal pigs: 0.54+/−0.16, adult humans: 1.41+/−0.5, adult pigs: 1.27+/−0.36 black/white pixels, neonatal humans vs. adult humans P=0.043, neonatal pigs vs. adult pigs P=0.035, neonatal human vs. neonatal pigs P=0.903, adult human vs. adult pigs P=0.938). Neonatal human and porcine samples resulted in higher scaffold fiber alignment values than adult humans or pigs (neonatal humans: 1.22+/−0.09, neonatal pigs: 1.14+/−0.07, adult humans: 1.09+/−0.02, adult pigs: 1.07+/−0.02; neonatal humans vs. adult humans: P=0.021, neonatal pigs vs. adult pigs: P=0.051, neonatal human vs. neonatal pigs: P=0.092, adult human vs. adult pigs: P=0.795).

Scaffold mechanical properties were examined using atomic force microscopy (AFM) (Asylum MFP3D-Bio, Asylum Research, USA) operated in force contact mode to obtain stiffness values. Plasma scaffolds formed with 0.5 U/mL human thrombin were polymerized directly on a glass slide 1.5 hours prior to force measurements. Standard silicon nitride cantilevers with a particle diameter of 1.98 μm (Nanoandmore, USA) were utilized. 20×20 μm force maps were collected on each scaffold and fit with a Hertz model to obtain the elastic modulus. A minimum of two random force maps were generated per scaffold with the average elastic modulus reported.

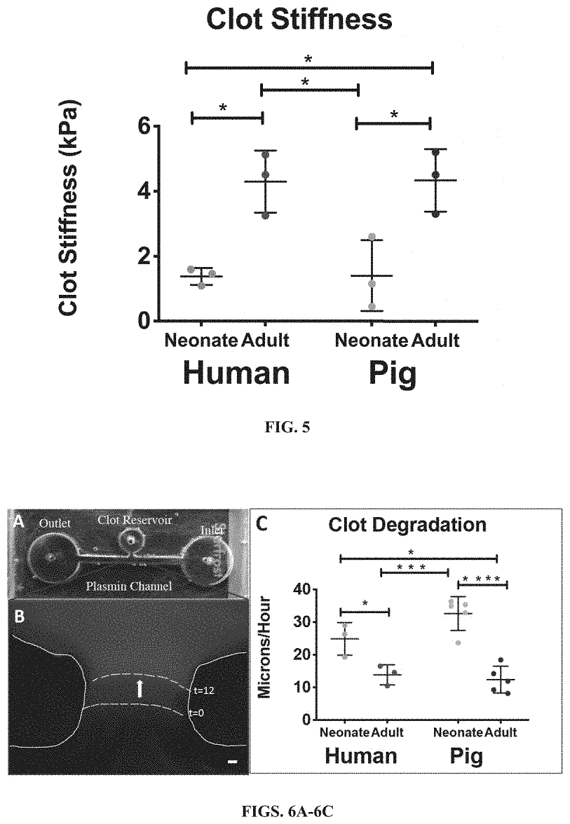

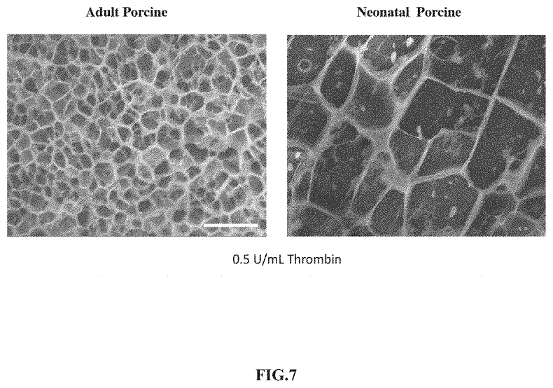

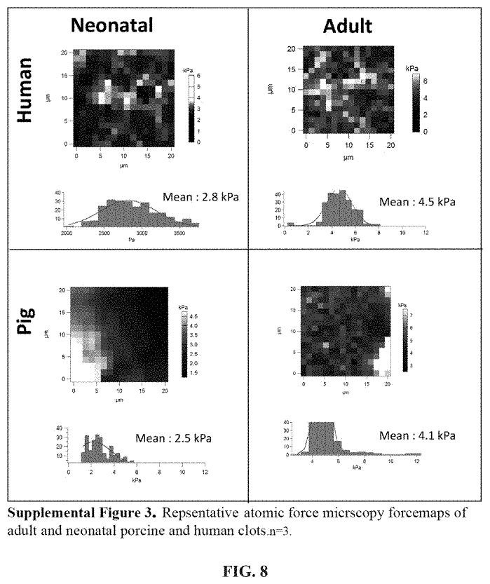

Fibrin scaffold mechanical properties were evaluated via. AFM Nano indentation. Force maps were generated (FIG. 8) and average stiffness values are shown in FIG. 5. Statistically significant lower scaffold stiffness values were observed in neonatal human plasma scaffolds compared to adults (p<0.05). These tendencies were mirrored in porcine samples (neonatal humans: 1.62+/−0.18, neonatal pigs: 1.4+/−1.1, adult humans: 4.3+/−0.95, adult pigs: 4.34+0.96 kPa; neonatal humans vs. adult humans: P=0.016, neonatal pigs vs. adult pigs: P=0.015, neonatal human vs. neonatal pigs: P>0.999, adult human vs. adult pigs: P>0.999).

Fibrinolysis was assessed for neonatal and adult human and porcine samples. A custom microfluidics-based assay was utilized to analyze degradation rates for all sample groups. A polydimethylsiloxane (PDMS) (Dow Corning, USA) device consisting of a scaffold reservoir with a perpendicular lying channel was constructed via casting in an acrylic mold. After curing for 24 hours, the device was plasma treated and bonded to a glass slide to create a sealed channel. Scaffolds were formed from plasma and 10% Alexa-Flour 488-labeled adult fibrinogen was added for visualization. Polymerization was initiated with the addition of 0.5 U/mL, of thrombin, and 25 μL of the scaffold solution was immediately injected into the scaffold reservoir. After polymerizing for two hours, the device was mounted on an EVOS FL Auto microscope (Life Technologies, USA) for imaging. A plasmin solution (0.01 mg/ml plasmin in HEPES buffer) (Human Plasmin, Enzyme Research Laboratories, USA) was injected into a channel inlet, and the scaffold was imaged every 10 min for 12 hours. ImageJ (National Institutes of Health, USA) was used to determine rate of scaffold degradation by comparing the first and final images and measuring distance along a perpendicular line to the scaffold boundary. Scaffold degradation rates were expressed as the distance the scaffold boundary traveled divided by 12 hours.

A custom microfluidics assay was utilized to determine plasma scaffold degradation rates (FIGS. 7A-7B) Scaffolds formed from neonatal human and pig plasma samples had significantly faster rates of degradation compared to adult human and pig groups (Neonatal humans: 24.9+/−4.9, neonatal pigs: 32.62+/−5.18, adult humans: 13.85+/−3.08, adult pigs: 12.38+/−4.11 μm/hour; neonatal humans vs. adult humans: P=0.048, neonatal pigs vs. adult pigs: P<0.0001, neonatal human vs. neonatal pigs: P=0.140, adult human vs. adult pigs: P=0.969).

The results show that age-related differences identified in human fibrinogen are mirrored in pigs, thus confirming piglets as an appropriate source for neonatal fibrinogen for creation of pro-healing fibrin-based scaffolds. To determine this, several aspects of fibrinogen and its resultant fibrin network were thoroughly analyzed across both species. It was found that fibrinogen concentration and functionality in plasma collected from piglets accurately parallels those observed in plasma collected from human neonates. Fibrin network structure, when analyzed via confocal and cryoSEM microscopy, also displayed similar tendencies with highly aligned fibrin networks in both neonatal species compared to highly branched networks in adults. Lastly, fibrin network stiffness and degradation patterns were assessed between neonates and adults in both species and again found substantial similarities.

To assess developmental similarities between human and porcine fibrinogen, a series of analyses comparing function, structure and degradation were conducted between adult and neonatal human and porcine fibrin networks. To analyze the functionality of fibrinogen between species, clottability for all sample groups were assessed and found statistically significantly lower clottability values in human neonatal fibrinogen than in adult fibrinogen. This same relationship was reflected in the porcine samples. Structurally, a three-dimensional, highly branched scaffold architecture was observed in adult porcine samples verses thin, sheet-like fibrin matrices with little cross branching in neonatal porcine samples. Quantitative analysis also revealed higher alignment and lower fiber density in neonatal porcine samples compared to adults. At a thrombin concentration of 0.5 U/mL, a comparative image analysis was performed. with human samples and again identified similar structural differences between age groups in pigs and humans. Both neonatal species exhibited fibrin scaffolds with a higher degree of alignment and statistically significant lower fiber densities when compared to corresponding adult scaffolds. Without wishing to be bound by theory, it is speculated that these structural differences are the result of differences in fibrin polymerization.

The disclosed validation also included analysis of human and porcine scaffold mechanical properties. Research has linked structurally dense, highly branched scaffolds to greater scaffold stiffness. Here. AFM was used to measure plasma scaffold stiffness in both porcine and human samples. Scaffolds formed from neonatal human plasma had statistically significantly lower average stiffness values than those formed from adult human plasma (p<0.05). These patterns were mirrored in scaffolds formed from neonatal and adult porcine samples (p<0.01).

In addition to polymerization rates, an accurate animal source must also display a fibrinolytic potential similar to neonatal humans. Thus, degradation rates were measured between age groups and species using a custom microfluidic device. statistically significantly faster plasma degradation rates were identified in neonatal samples compared to adults. Both neonatal porcine and human plasma scaffolds exhibited rapid rates of degradation and lower fiber densities when compared to adult samples.

The simplified system utilized in the study allowed for the detailed focus on age dependent differences in fibrinogen and plasma fibrin networks between humans and pigs. Also, due to the accelerated timeline of ageing in pigs compared to humans, it is likely the eight-week old piglets used in this study do not accurately reflect the neonatal period in humans. The samples utilized in this study were obtained based on availability from NCSU's School of Veterinary Medicine. It was shown that 8-week old piglets could serve as a useful preclinical model of human neonatal fibrin deficiencies. Also, using 8-week old piglets is logistically easier than very young, newborn pigs as they are already weaned. Additionally, all sample groups utilized herein included both male and female samples except for the adult porcine group in which there were only females. This study utilized plasma samples from healthy piglets in order to establish a baseline in vitro model.

In summary, the results validate that piglets can serve as an appropriate animal model capable of reflecting the developmental nuances observed in human fibrinogen. Recent evidence confirms that neonatal fibrinogen is qualitatively distinct from adult fibrinogen resulting in differences between neonatal and adult fibrin scaffold structure. It was observed similar fibrinogen concentrations and clouability across species as well as similar age-related pattern is in structure, mechanical, and degradation properties of adult and neonatal porcine and human samples. Based on these results, piglets are indeed an appropriate animal source for neonatal fibrinogen for creating pro-healing fibrin-based scaffolds.

### Example 2: Comparison of Wound Healing Properties of Fibrin Scaffolds Derived from Adult Blood with Fibrin Scaffolds Derived from Neonatal Blood

Neonatal and adult human fibrinogen was isolated from plasma samples via ethanol precipitation reaction. After IRB approval from Emory University and informed written parental consent, whole blood samples were collected from human neonates (less than 30 days of age) undergoing elective cardiac surgery at the Children's Hospital of Atlanta. 5 mL of whole blood was collected from an arterial line placed after the induction of anesthesia and prior to surgical incision. Samples were centrifuged immediately to yield PPP and stored at −80 until use. Pooled adult human PPP was obtained from the New York Blood Center and stored at −80 until use. Pooled plasma samples were then utilized for isolation of either adult or neonatal fibrinogen. Ethanol (70% volume) was added to 4° C. plasma in a 4:1 ratio (plasmalethanol) and cooled on ice for 20 minutes. The solution was centrifuged at 600 g for 15 minutes at 4° C. The supernatant was then removed, and the resulting pellet is heated in a 37° C. water bath. A buffer consisting of 20 mM sodium citrate was added until the pellet was completely dissolved (0.25-0.5 mL). This method mainly selectively precipitates fibrinogen, however, trace amounts of other plasma proteins including Von Willebrand Factor, plasminogen, and fibronectin may be present. If necessary, fibrinogen solution was concentrated using Pall Nanosep centrifugal devices (Pall, Port Washington, N.Y., USA). Concentration of samples was determined using a Nano-drop (Thermofisher Scientific, Waltham, Mass., USA). For all in vitro assays, conditions were run with a minimum of two separate isolated fibrinogen batches. For in vivo studies, a single isolation hatch was used.

### Structural Characterization of Fibrin Matrices

Structural analysis of fibrin scaffolds was conducted with confocal microscopy. 50 μl scaffolds consisting of purified adult or neonatal fibrinogen at a concentration of 2.5 mg/mL in HEPES buffer (5 mM calcium, 7.4 pH) were formed with the addition of 0.5 U/mL human α-thrombin. 10 μg/mL Alexa 488 labeled fibrinogen was used for visualization. Scaffolds were formed between a glass slide and coverslip and allowed to polymerize for two hours prior to imaging. A Zeiss Laser Scanning Microscope (LSM 710, Zeiss Inc., USA) at a magnification of 63× was utilized for imaging and a minimum of three random z-stacks of 5.06 μm thickness were acquired per scaffold. ImageJ software was used to create 3D projections from z-stacks. Fiber alignment was quantified through a MATLAB algorithm previously utilized by our group. The code can be found at haps://github.com/Kniellen/Fiber-alignment. Briefly, each confocal microscopy image was preprocessed by padding with redundant data and applying a gaussian decay and two dimensional Hann window to minimize edge effects prior to application of a two-dimensional Fast Fourier transform. The resulting power spectrum was utilized to determine alignment by polar coordinate analysis and relative intensity of pixels in angular bins. The alignment index (AI) was determined from the fraction of fibers aligned within +/−20 degrees of a preferred fiber alignment normalized to random distribution of oriented fibers (equal to 40°/180°). Alignment index values range from 1.0 to 4.55. Alignment analysis was conducted for each image in the z-stack and averaged together. Scaffold fiber density was determined from the ratio of black (fiber) over white (background) pixels in each image. Fibrin network branching was quantified with a custom MATLAB code. Briefly, a multiscale-hessian filtering method was applied to each image slice to identify tubular structures. Structure sensitivity was determined through a threshold based on the intensity distribution. The image was than binarized and skeletonized. Fiber overlap was then quantified from the skeletonized image by reducing intersections of fibers to single points. These branch points were normalized across the area of the image and averaged across each stack. Each image of the three-dimensional stack was processed individually and then averaged together.

### Removal of sialic acid from Fibrinogen

To determine the influence of sialic acid on neonatal and adult fibrin structure, sialic acid was cleaved from neonatal and adult fibrinogen via neuraminidase. A 500 μl fibrinogen solution consisting of 5 mg/ml adult or neonatal fibrinogen in diH₂O was incubated with 0.025 U Neuraminidase (Neuraminidase/Sialidase, Sigma Aldrich, USA) for 4 hours at 35° C. Removal of bound sialic acid was confirmed by determining the concentration of sialic acid in the fibrinogen solution before and after enzyme digestion (Sialic Acid (NANA) Assay Kit, Abcam, USA). The solution was centrifuged and stored at −80° C. until use in confocal microscopy experiments.

### Cell Attachment an Fibrin Matrices

Cell attachment on fibrin matrices was analyzed via a florescence based assay. Neonatal and adult fibrin networks (2.5 mg/mL) were formed with 0.5 U/mL human α-thrombin (Enzyme Research Laboratories, South Bend, Ind., USA) in a 96-well plate and polymerized for 2 hours. Neonatal human dermal fibroblasts (HDFn) (Gibco, Walthani, Mass., USA) (P5-P14) were fluorescently labeled (Vybrant Dii, Thermofisher Scientific, Waltham, Mass., USA) according to manufacture instructions, seeded on top of fully polymerized fibrin gels, and incubated for 1 hour at 37° C. After 1 hour, three washes with PBS were performed to remove non-adherent cells and florescence intensity (Abs:549 nm, Em: 565 nm) was determined via plate reader (Biotek Synergy H1). It is possible that fluorescence intensity may vary from cell to cell, therefore, cell attachment was also quantified from confocal microscopy images taken at 40× from cells seeded at a density of 12,000 cells/well and fixed after 16 hours. Cell count was determined as the number of cells in the field of view. The average values from 12 images at the same magnification are reported.

### Cell Spreading on Fibrin

Cell spreading on fibrin films was analyzed via confocal microscopy. Uniform fibrin films were formed on coverslips using modified standardized protocols. To create hydrophilic bottom coverslips to adhere to fibrin matrices, a 0.1 M sodium hydroxide (NaOH) solution was added to coverslips in a 12 well cell culture plate and left to evaporate. The coverslips were then functionalized with (3-Aminopropypiriethoxysilane (APTMS) for 5 minutes at room temperature. Once dry, a 0.05% glutaraldehyde solution was added to each well and left to incubate for 30 minutes followed by 3 washes with H₂O. Neonatal and adult fibrin gels consisting of 2.5 mg/mL fibrinogen and 0.5 U/mL human α-thrombin were then formed on functionalized coverslips and immediately covered with dichlorodimethylsilane (DCDMS) coated coverslips. After 2 hours of polymerization, top coverslips were removed and fibrin gels were stored at 4° C. until use. Gels were sterilized via UV light for 30 mins prior to cell attachment. HDFns were seeded on fibrin gels at a density of 12,000 or 6,000 cells per well and media (HDFn growth medium; DMEM, 10% fetal bovine serum, 1% pencillin-streptomycin, 1% L-glutamine); up to 1 mL was added to each well. Plates were then incubated for 16 hours at 37° C. Cells were fixed with 4% paraformaldehyde in PBS and stained with Alexa 488 labeled phalloidin for visualization. Samples were mounted with Vectashield with Dapi (Fisher Scientific, Hampton, N.H., USA) to visualize nuclei and coverslips were sealed with nail polish. Confocal microscopy (Zeiss LSM 710) was utilized to examine cell morphology and cell area, perimeter, and circularity were calculated with ImageJ.

### Evaluation of In Vitro Cell Migration in Neonatal vs. Adult Librin Matrices

HDFns were cultured into spheroids over 72 hours using a hanging drop cell culture technique. Fibrin scaffolds composed of 2.5 mg/ml human neonatal or fibrinogen and 0.5 U/ml human α-thrombin (Enzyme Research Laboratories, South Bend, Ind., USA) were created in the wells of a 96-well tissue culture plate (VWR, Radnor, Pa., USA). After a 2 hour polymerization period, cell spheroids were transferred onto a fibrin scaffold using a 21 G×1½′ needle (BD Biosciences, San Jose, Calif., USA) and then covered with a second fibrin layer to create a 3D environment. After a 2 hour polymerization time for the second fibrin layer, HDFn growth medium (DMEM, 10% fetal bovine serum, 1% penicillin-streptomycin, 1% L-glutamine) was added into each well, and spheroids were imaged every 24 hours for a 72-hour period. Cell migration throughout the fibrin matrix was quantified by measuring the projected spheroid surface area for each 2D image using ImageJ.

### Evaluation of Wound Healing In Vivo

Wound healing in vivo in the presence of neonatal and adult fibrin scaffolds was assessed using a murine wound healing model previously described by Dunn ct al. All protocols were approved by the NCSU IACUC prior to conducting studies. 8 week old C57/B6 mice (Charles River Laboratories, Wilmington, Mass., USA) were anesthetized with 5% isoflurane in oxygen in an induction box prior to surgery. Throughout the duration of the surgery', general anesthesia was maintained with a nosecone at 3% isoflurane. Full thickness dermal wounds were created using 4 mm biopsy punches (Fisher Scientific, Hampton, N.H., USA) and splinted with 10 mm silicone rings with a 5 mm inner diameter. Wounds were splinted to force healing through reepithelization rather than skin contraction as it is more physiologically relevant to humans. 10 μL of neonatal or adult fibrin scaffold treatments created with 2.5 mg/mL fibrinogen polymerized in the presence of 0.5 U/mL human α-thrombin were applied topically with 0.9% sterile filtered saline as a control group (n=6 wounds/group). Wounds were imaged and covered with Opsite bandages (Fisher Scientific, Fiainpton, N.H., USA). Carprofen (ApexBio, fiouston, Tex., USA) (5 mg/kg) was administered subcutaneously for pain relief for the first five days post-surgery. Wounds were imaged and dressings were changed every day for nine days post-surgery. Wound size analysis was performed on wound images that were blinded by treatment group and randomized using a random number generator. Wound sizes were quantified using ImageJ and normalized to the silicone ring openings. Normalized wound areas were used to determine wound closure rates for each treatment group. Total wound healing rate was calculated as the total percent wound closure on day 9 divided by 9.

Animals were euthanized under carbon dioxide and then tissue surrounding the wounds was excised nine days post-surgery and fixed in 10% formalin. Tissue samples were ethanol dehydrated, embedded in paraffin wax, and sectioned for analysis. Martius Scarlet Blue (MSB) staining was performed to identify collagen and fibrin within the wound site. Epidermal thickness was quantified from MSB images by measuring the thickness of the epidermal layer at 3 regions across the wound area. CD31 labeling was performed to evaluate angiogenesis within tissue forming at the wound sites. Tissue was deparaffinized, rehydrated, and incubated with 1% goat serum (Thermo Fisher Scientific, Waltham, Mass., USA). Sections were labeled with a rabbit anti-mouse monoclonal antibody to CD31 (1:50, clone SP38, Thermo Fisher Scientific, Waltham, Mass., USA) overnight at 4° C. Sections were then washed in PBS and labeled with Alexa 594 goat anti-rabbit as a secondary antibody for one hour at room temperature. Sections were then washed in PBS and mounted with Vectashield HardSet mounting medium with DAPI (Fisher Scientific, Hampton, N.H., USA). CD31 positively labeled tissue was quantified. Briefly, ImageJ Particle Analysis was used to measure total red area (defined as red area greater than 1.0 μm² with a threshold of 0-50) in wounds.

### Statistical Analysis

Statistical analysis was performed in GraphPad Prism 7 (GraphPad, San Diego, Calif., USA). Data was analyzed via a One-way Analysis of Variance (ANOVA) with a Tukey's post hoc test using a 95% confidence interval for all measurements except for analysis of wound closure, which was conducted with a two way ANOVA. Outlier tests were performed prior to all statistical analysis. No outliers were found and no data was removed. Data is presented as average+/−standard deviation.

### Results

### Structural Characterization of Fibrin Matrices

Here, we utilized confocal microscopy to compare the architecture of fibrin scaffolds constructed from neonatal or adult fibrin (FIG. 9A), At equal fibrinogen (2.5 mg/mL) and thrombin (0.5 U/mL) concentrations we observed major structural distinctions between scaffolds. Specifically, adult fibrin networks were found to have a dense, branched structure compared to the highly porous, low cross branching structure seen in neonatal networks. Analysis of fiber density using the ratio of black to white pixels revealed significantly lower fiber density in neonatal samples compared to adult scaffolds (FIG. 9B) (Neonatal fibrin: 0.684+/−0.157, Adult fibrin: 1,350+/−0.201, p<0.0001). Using a custom MATLAB code for fiber alignment we observed a higher, although not statistically significant, degree of fiber alignment in the neonatal samples compared to adult (FIG. 9C) (Neonatal fibrin: 1.095+/−0.073 alignment index, Adult fibrin: 1.054+/−0.030 alignment index, p=0.142). Quantification of fibrin branching indicated significantly more branch points per area in adult samples compared to neonate (FIG. 9D) (Neonatal fibrin: 0.045+/−0.006 branch points/μm², Adult fibrin: 0.057+/−0.010 branch points/μm², p<0.01).

### Cells Display Enhanced Attachment, Spreading, and Migration on Neonatal Fibrin Scaffolds

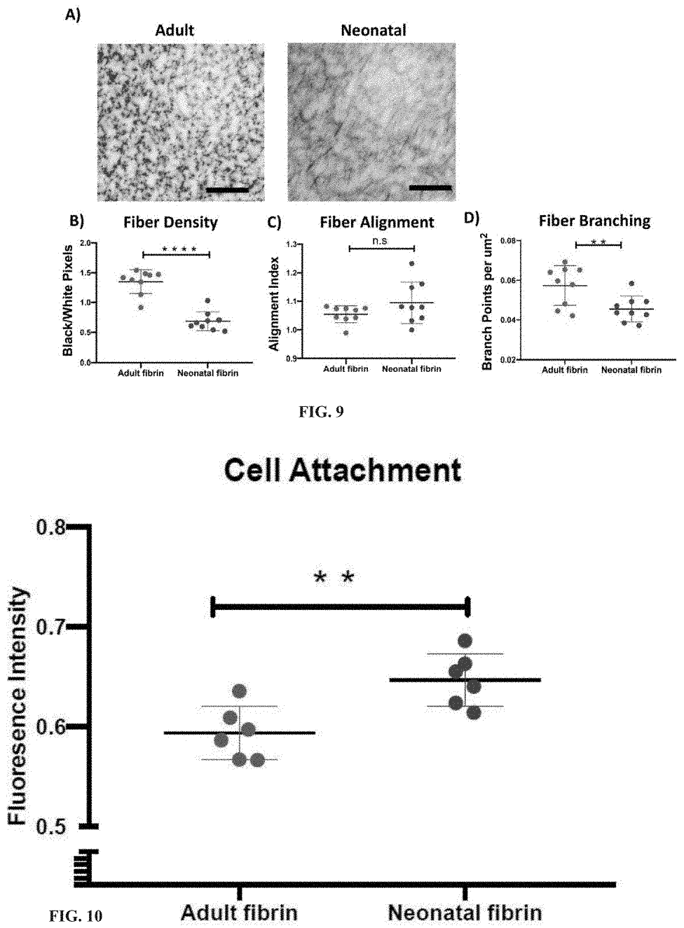

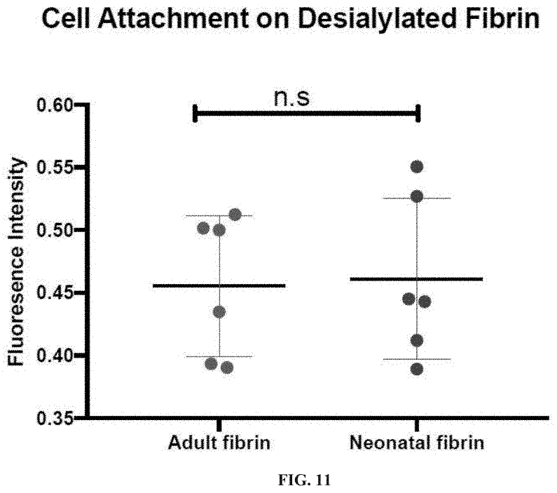

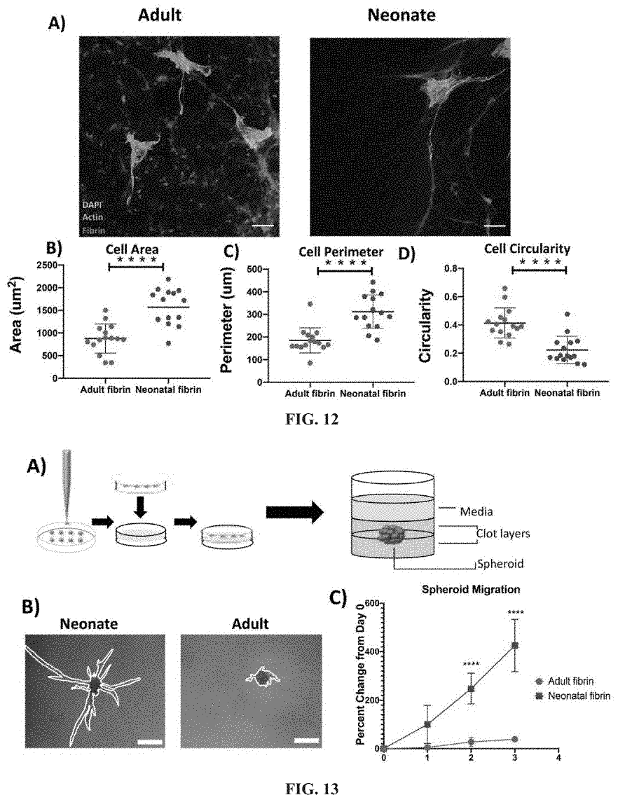

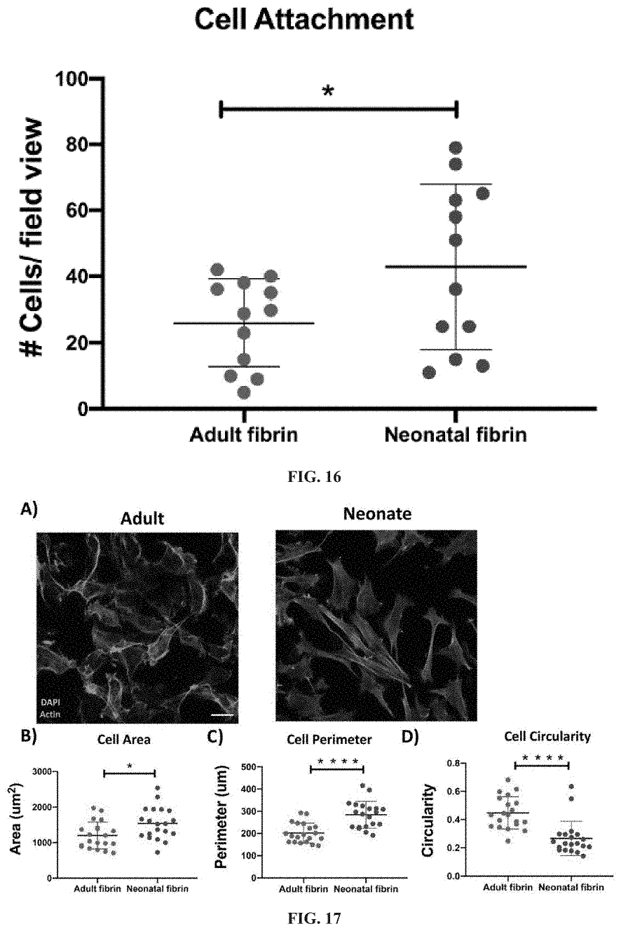

Fibroblast attachment to the provisional fibrin matrix is a crucial step in wound healing and is mediated by surface integrin αvβ3. Fibrin architecture is known to influence fibroblast attachment, spreading, and migration, therefore we hypothesized that the differences observed in adult and neonatal fibrin scaffolds would likewise differentially influence fibroblast behavior. To that end, fibroblast attachment on neonatal or adult fibrin scaffold was first explored with fluorescent) labeled fibroblasts (FIG. 10). After the 1 hour incubation period, attachment of fibroblasts on neonatal fibrin matrices was found to be significantly higher than on adult fibrin (Neonatal fibrin: 0.647+/−0.027 fluorescence intensity, Adult fibrin: 0.594+/−0.026 fluorescence intensity, p<0.01). Additionally, cell attachment was determined from cell counts on images taken after 16 hours of incubation (FIG. 16). Similarly, cell attachment on neonatal fibrin matrices was higher than on adult fibrin matrices in these experiments (Neonatal fibrin: 42.91+/−24.92 cells/field of view, Adult fibrin: 26.00+/−13.21 cells/field of view, p<0.05). While the underlying specific cause of structural differences between neonatal and adult fibrin networks remains a topic of investigation and is likely multifactorial, these differences could be due to differences in posttranslational modifications. Previous studies have identified differences in sialic content between adult and neonatal fibrinogen, therefore, we next investigated the influence of sialic acid on the observed cell attachment responses by removing sialic acid from the fibrinogen prior to fibrin polymerization and then evaluated cell attachment. When sialic acid was removed from both adult and neonatal fibrinogen with neuraminidase incubation, no significant differences in fibroblast attachment were observed on fibrin scaffolds, indicating the presence of sialic acid may influence cell attachment (FIG. 11) (Neonatal fibrin: 0.466+/−0.064 fluorescence intensity, Adult fibrin: 0.455+/−0.056 fluorescence intensity). We next characterized cell morphology of adherent fibroblasts on neonatal and adult fibrin scaffolds with confocal microscopy at 6,000 cells/well (FIG. 12) and 12,000 cells/well (FIG. 17). At various cell densities, cell area and perimeter were increased with decreased cell circularity on neonatal fibrin scaffolds compared to fibroblast parameters on adult fibrin matrices (6,000 cells/well; cell area; Neonatal fibrin: 1571.09+/−397.82 μm² Adult fibrin: 877.12+/−323.02 μm², p<0.0001, cell perimeter: Neonatal fibrin: 311.99+/−73.95 μm, Adult fibrin: 184.94+/−55.45 μm, p<0.0001, cell circularity; Neonatal fibrin: 0.223+/−0.09, Adult fibrin: 0.4138+/−0.11, p<0.0001). Finally, we evaluated fibroblast migration through 3D neonatal or adult fibrin scaffolds using a spheroid based assay. Fibroblast migration through fibrin networks was observed over a 72 hour period (FIG. 13). Cell migration was found to be significantly greater at 48 and 72 hours on neonatal derived fibrin scaffolds compared to adult (48 hours: Neonatal fibrin: 248.16+/−63.29% change from day 0, Adult fibrin: 27.75+/−18.27% change from day 0, p<0.0001, 72 hours: Neonatal fibrin: 425.61+/−107.89% change from day 0, Adult fibrin: 38.57+/−11,68% change from day 0, p<0.0001).

### Evaluation of Wound Healing In Vivo

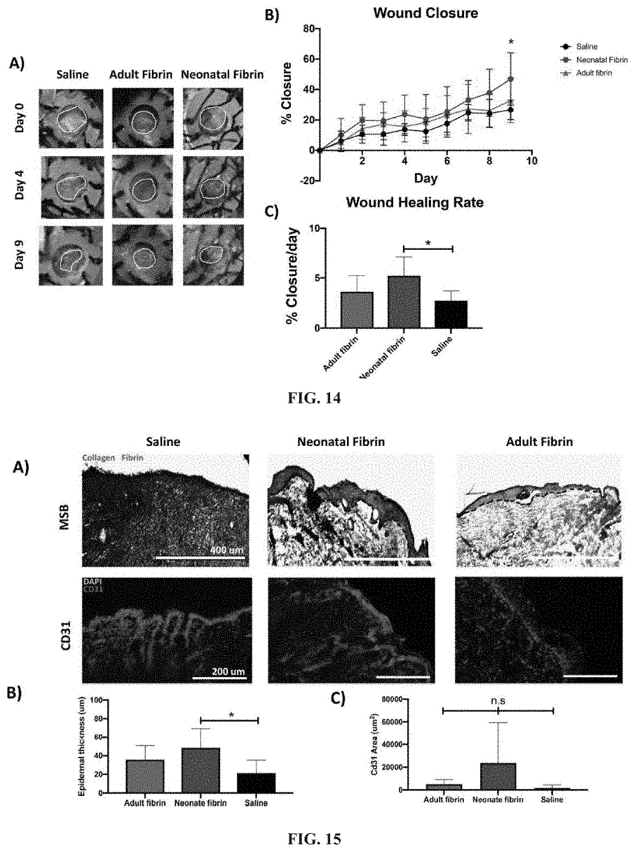

Fibroblasts migrate into the provisional fibrin matrix and synthesize new extracellular matrix during native wound healing to form new tissue. Based on our results demonstrating enhanced cell attachment and migration on neonatal fibrin scaffolds compared to adult scaffolds, we hypothesized that wound healing outcomes would also be improved. To assess this, neonatal or adult fibrin scaffolds (2.5 mg/mL fibrinogen, 0.5 U/mL thrombin) or a saline control were applied to full thickness dermal wounds in adult mice. Rates of wound closure was assessed by measuring the wound area over the 9 day period. Evaluation of wound closure indicated an overall greater rate of closure and significantly smaller wound areas on day 9 in mice treated with neonatal fibrin compared to adult fibrin and saline treatment groups (wound closure rate; Neonatal fibrin: 5.21+/−1.91% closure/day, Adult fibrin: 3.628+/−1.605% closure/day, Saline 2.72+/−0.987% closure/day, Neonatal fibrin vs. Saline p<0.05) (FIG. 14). Histological analysis of wound tissue stained with MSB staining revealed a more robust epithelial layer with significantly greater thickness in neonatal fibrin treated wounds compared to wounds from mice treated with a saline control (Neonatal fibrin: 48.33+/−20.84 μm, Adult Fibrin: 35.67+/−15.03 μm, Saline: 21.27+/−13.86 μm. Neonatal fibrin vs. Saline p<0.05) (FIG. 15). Immunohistochemistry staining for angiogenic marker CD31 indicated increased angiogenesis in wounds treated with neonatal fibrin relative to wounds treated with adult fibrin or saline, although statistical significance was not reached (Neonatal fibrin: 23630.00+/−35634.40 μm², Adult Fibrin: 5040.5+/−4057.33 μm², Saline: 1744+/−2687.34 μm²).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and. so forth used in the specification and claims are to be understood at the very least, and not as an attempt to limit the application of the doctrine of equivalents to the scope of the claims, to be construed in light of the number of significant digits and ordinary rounding approaches.

1. A fibrin scaffold comprising polymerized fibrinogen, where the fibrinogen is obtained from an animal less than 18 months of age.

2. (canceled)

3. The fibrin scaffold according to claim 1, wherein the scaffold is platelet free.

4. The fibrin scaffold according to claim 1, having an average stiffness of less than 5 k*Pa.

5. (canceled)

6. The fibrin scaffold according to claim 1, having a fiber density less than 2, as measured by confocal microscopy.

7. (canceled)

8. The fibrin scaffold according to any of claims 1-7, having a fiber alignment index of at least 0.5.

9. (canceled)

10. The fibrin scaffold according to claim 1, wherein the fibrinogen obtained from an animal less than 18 months of age has a clottability of less than 99.

11. (canceled)

12. The fibrin scaffold according to claim 1, wherein the fibrinogen is polymerized by thrombin.

13. (canceled)

14. The fibrin scaffold according to claim 1, wherein the animal is a pig.

15. The fibrin scaffold according to claim 1, in the form of particles.

16. (canceled)

17. The fibrin scaffold according to claim 1, in the form of particles having a particle size no greater than about 500 nm.

18. The fibrin scaffold according to claim 15, wherein when dehydrated, the particles have a height that is no more than 20% of the particle width.

19. (canceled)

20. The fibrin scaffold according to claim 1, having a degree of crosslinking that is not greater than 25%.

21. (canceled)

22. (canceled)

23. The fibrin scaffold according to claim 1, further comprising at least one therapeutic agent.

24. The fibrin scaffold according to claim 23, wherein the at least one therapeutic agent comprises one or more antimicrobials, analgesics and anti-inflammatories.

25. A method of treating or healing a wound, comprising contacting the wound with the fibrin scaffold according to claim 1.

26. The method of claim 25, wherein the wound is directly contacted with the fibrin scaffold.

27. A method of preparing the fibrin scaffold according to claim 1, comprising polymerizing a platelet deficient fibrinogen with thrombin.

28. The method of claim 27, wherein the polymerization occurs at the location of a wound.

29. A method of treating or healing a wound, comprising contacting the wound with a first composition comprising platelet poor fibrinogen and a second composition comprising thrombin, to produce a fibrin scaffold according to claim 1.

30. The method according to claim 29, wherein the first composition further comprises at least one therapeutic agent, the second composition further comprises at least one therapeutic agent, or both the first and second compositions comprise at least one therapeutic agent.
